(19) United States
(12) Patent Application Publication
(10) Pub. No.: US 2005/0250736 A1
(43) Pub. Date: Nov. 10, 2005

(54) Composition and method for treatment of obesity and smoking cessation

(76) Inventor: Michael Cherkassky, Fort Worth, TX (US)

Correspondence Address: MICHAEL CHERKASSKY M.D., 1550 W. Rosedale # 418, Fort Worth, TX, 76104, US

(21) Appl. No.: 10/841,920

(22) Filed: May 10, 2004

(51) Int. Cl. A61K 31/716; A61K 9/68; A61K 47/00

(52) U.S. Cl. 514/57; 424/439; 424/48

(57) ABSTRACT

The invention involves an instrument of weight loss by means of appetite suppression, along with a method for administering this preparation to humans and other mammals. The preparation consists of a variety of different spices, sweeteners, and salts, as well as proteins and alcohols. Additionally, there is a remedy for smoking cessation that consists of the application of the above ingredients to the tongue, in combination with the subsequent use of tobacco extracts drops.

### REFERENCES

Bhavar S, Watkins J, Young A. Synergy between amylin and cholecystokinin for inhibition of food intake in mice. Physiol Behav. 1998;42:45-50.
Heineman L, Heise T, Wahl L C, Trautmann M E, Ampudia J, Starke A A, Berger M. Prandial glycaemia after a carbohydrate-rich meal in type 1 diabetic patients: using the rapid acting insulin analogue [Lys(B29)] human insulin. Diab Med. 1996;13:625-629.
Kahn S E, Fujimoto W Y, D'Alessio D A, Ensinck J W, Porte D Jr. Glucose stimulates and potentiates islet amyloid polypeptide secretion by the B-cell. Horm Metab Res. 1991;23:577-580.
Reidelberger R D, Arnelo U, Granqvist L, Permert J. Comparative effects of amylin and cholecystokinin on food intake and gastric emptying in rats. Am J Physiol Regul Integr Comp Physiol. 2001;280:R605-611.
Schwartz M W, Woods S C, Porte D Jr, Seeley R J, Baskin D G. Central nervous system control of food intake. Nature. 2000;404:661-671.
Williams B, Werner J, Holst J J, Orskov C, Creutzfeldt W, Nauck M A. Gastric emptying, glucose responses, and insulin secretion after a liquid test meal: effects of exogenous glucagons-like peptide-1 (GLP-1)-(7-36) amide in type 2 (noninsulin-dependent) diabetic patients. J Clin Endocrinol Metab. 1996;81:327-331.

### BACKGROUND—FIELD OF INVENTION

The present invention relates to food and tobacco products composed and arranged in such a way that is it appropriate for weight maintenance or weight loss, and provides therapeutic benefits for mammals, as well as enables patients to overcome tobacco addition.

### BACKGROUND—DESCRIPTION OF PRIOR ART

Obesity is one of the most major problems within the Western community today. Due to increased consumption and changes in nutritional value, many humans and companion animals suffer from being overweight. Today, obesity is one of the most serious health problems in the United States, with approximately 30% of the adults in the country suffering from obesity and at least 50% of the adults in the United States who are overweight. The problem of obesity in the United States and most western countries has been steadily increasing in the last several decades. Such obesity has caused or contributed to a marked increase in the occurrence of heart diseases, hypertension, diabetes, arthritis, and increased morbidity and mortality. There is also recent research that links obesity with different types of cancer, particularly breast cancer. Obesity is a serious public health hazard, second in importance only to tobacco. The condition of being overweight reduces one's lifespan as well as quality of life.

There are many methods of suggested management of obesity and in being overweight. They include diets that prohibit fats and high caloric elements, appetite suppressants, a number of psychotheraputic techniques, and even a variety of operative techniques. One of the most common methods is the use of the stimulants: amphetamine-like agents that act on the brain to reduce the sensation of hunger. Experience indicates that most of the appetite suppressants work for a short period of time—‘a few weeks or a few months’—after that, they lose most of their potency and, unfortunately, patients begin regaining the lost weight.

There is also a serious problem with the maintenance of a desirable level of a person's weight after it is achieved for a simple reason: appetite suppressants cannot be continued indefinitely.

The hazards of tobacco smoking from chemicals such as coal tars, carbon monoxide, and nicotine have been vastly researched and are well known. The majority of lung cancers are direct results of smoking tobacco, as well as cancers of the urinary tract, such as bladder cancer, and a variety of heart and circulation problems, such as high blood pressure.

There is an enormous number of Americans who try to stop smoking annually. Most of them do not succeed. The difficulties in ending the use of tobacco are great. The nicotine withdrawal syndrome presents itself in the many following negative effects: increased tensions, irritability, restlessness, intense cravings, depression, bradicardia, hypotention, constipation, sleep disturbance, and, finally, an increase in body weight.

The reason most people become overweight is that they consume more nutritional ‘calories’ than is required of them. The abundance of food and the fact that food is readily available, as well as relatively inexpensive in the developed countries, results in inordinate consumption. Thus, consumption is regulated not by necessity, but by the satisfaction of the palate. In other words, we eat not to survive, but for the taste, flavor, and gratification.

The degree of the satiety has changed and is not dependant upon the necessary nutritional requirements, but on the ‘unnatural’ drive to pleasure.

Multiple interdependent processes characterize complex mechanism, which is triggered in a mammal's body in the process of food digestion, where various hormones with multiple functions influence different organs at the same time. Due to this process, the whole body may function properly.

One of the necessary sensations, which completes the mechanism of digestion, is satiety. There are many unknowns in our knowledge of satiety, but what is known to us indicates the number of hormones needed to participate in the process of digestion also plays a significant role in inducing a sense of this satiety.

There is a group called gastrointestinal peptides. The predominance of polypeptides are produced in and secreted from specialized gut endocrine cells as well as nerves.

The production of gastrointestinal hormones increases when food, intraluminal pH, releasing factors, and other transmitters or hormones stimulate gut endocrine cells. There is a number of fairly well known gastrointestinal hormones such as the following: Amylin, CCK, Gastrin, Secretin, Enterostatin, and polypeptide Y 3-36. All of these hormones play a specific role in the digestion processes in the intestinal tract, but they also participate in transmitting information to the brain. This enables the brain to be well-appraised of the quantity and quality of food being consumed, and, therefore, regulates the amount of food intake from meal to meal.

Information from the gastrointestinal tract and oropharynx is newly transmitted to the brain.

It has been proven that these hormones reach “tractus solitarius” via the hypothalamus, and concentrated in this area, they induce satiety. Among these hormones are CCK and Amylin, and possibly Insulin.

Amylin and CCK also reduce gastric emptying and intestinal mobility, thus delaying the delivering of food to the intestines and contributing to the early sense of the satiety. As a result, they limit the quantity of food being consumed.

The above description illustrates that there are some mechanisms triggered by food consumption that also play a role in limiting the quantity of food being consumed during one particular meal.

It is postulated by the author of this patent that by using certain food ingredients with limited nutritional value—but capable of stimulating the intestinal tract—it will be possible to reach the early stages of satiety.

There are a number of ingredients in foods that fit this description. They are: pepper, mustard, cinnamon, sugar, sugar substitutes, salt, alcohol, proteins, and albumins cellulose.

### SUMMARY OF THE INVENTION

It is the objective of the present invention to develop a composition of the aforementioned ingredients that can effectively act to control the weight of the mammal to which the composition is administered. It is a further objective of the present invention to develop a composition of food products that can act to control the weight of a mammal to whom the composition is administered. This composition suppresses appetite as a result of inducing early sense of the satiety. In addition, it is the objective of the present invention to develop a method of administering a composition of food products to control weight without notable side effect.

These and other objectives are partly achieved by a method of administering a composition of pepper, mustard, sugar, salt, alcohol, proteins albumins, cellulose ether, and a variety of other spices, sweeteners, and salts to a mammal—preferably human in manner—to effectively control the weight of the mammal.

In this method, it is preferable to administer the medication orally. This objective is achieved by ingesting a weight control composition comprised of different food ingredients containing at least some spices, sweeteners, salts, and proteins.

### OBJECTS AND ADVANTAGES

1. The application of the composition and the methods are easy to manufacture.
2. The administrator of the composition does not require extensive training.
3. The composition is easy to manufacture.
4. The composition could be used in conjunction with all other available means, including prescription drugs, as well as exercise.
5. The patient is not restricted to or from any particular type of food (artificial food). There is merely a caloric restriction.
6. The methods can be used for a longer period of time than any other weight loss medication.
7. Due to the fact that the method and composition has virtually no side effects, it is ideal for maintenance (long-term use).
8. The methods and composition that are utilized for smoking cessation are easy to apply and do not involve any long-term negative side effects.
9. The composition has virtually no negative side effects.

### DESCRIPTION—PREFERRED EMBODIMENT

The preferred embodiment of the invention involves the administration of a variety of specially combined food ingredients with limited nutritional value, which singularly, or in a combination, is capable of stimulating intestinal hormones. These hormones, in turn, would influence the brain specifically in ‘tractus solitarius,’ and induce early satiety. This would, therefore, drastically limit the quantity of food consumed at any particular meal. The same intestinal hormones also work to reduce gastric emptying, resulting to the same goal as of limiting food consumption.

Dosages and Preparations

The single oral dosage consists of 0.8 cc capsule with the following components: approximately 87% of cellulose ether, 10% of proteins, 0.1% of pepper, 0.1% of saccharin, 0.2%, of mustard, 2% salt, and 0.5% of alcohol. The proportions of the ingredients could vary and other similar food ingredients could be included. Initially, the entire preparation is thoroughly mixed and evaporated in the microwave. Obviously, some other temperature treatments could be applied, including the utilization of a stove or a freezer.

There are three types of preparations. The first is the basic one described above. Another preparation has the inclusion of 200 mg of caffeine. This is designed to be used in the early part of the day. A third preparation has approximately 100 mg of caffeine, and is used in the later part of the day. The reason for the caffeine inclusion is that during the weight loss processes, people frequently feel exhausted and tired, and need a certain boost of energy.

It is interesting to note that the basic, caffeine-free preparation is most convenient for the night time use because, at present, there are basically no appetite suppressants readily available which are not stimulants. As mentioned before, with a few exceptions, appetite suppressants are stimulants by nature and cannot be used before sleeping hours. This roadblock leads to a tremendous gap in protection for the patient.

Another interesting aspect of these new discoveries is that non-caffeine preparations could be used to enhance the appetite suppressant effects of the caffeine-containing preparations, as well as other appetite suppressors including prescription drugs. This means that it is possible to extend the appetite suppression effect to a much higher degree then has been previously available.

Yet another advantage of this invention is that this new discovery could be combined with any other commercially available appetite suppressants—or, for that matter, anything which will be discovered in the future—because this particular preparation has virtually no side effects.

Another embodiment of the above principles could be applied, utilizing the close connection between the tongue, oral cavity, oropharynx, and intestines. When a small tablet is placed in the mouth without being swallowed, it triggers a similar chain of events that result in early satiety. The tablet consists of the combination of different spices, sweeteners, and salts.

An extension of the above idea could be an application of the paste (consisting of spices, sweeteners, and salt) on select parts of the tongue with the simultaneous feeding of small quantities of food products smeared with the paste. The food products would, naturally, be presented as the ones patients crave most. In the case of a liquid product, the composition is administered as a powder, and mixed into the liquid. In this case, there will still be the same paste application to the tongue. Therefore, this would allow the patient to eliminate or greatly reduce the dependence on the products they crave.

Yet another positive is that the aforementioned paste could be utilized for smoking cessation. One would apply the paste on the part of cigarette, which is placed in the mouth, and then the patient would smoke. The patient would, therefore, inhale through the thick coat of paste. Prior to this, though, the application of the paste should be administered on the tongue.

After the above procedure, the patient should be supplied with tobacco extract suspended in alcohol, and instructed to use it in the form of drops whenever he or she feels the urge to smoke.

### CONCLUSIONS, RAMIFICATIONS AND SCOPE OF THE INVENTION

Accordingly, the reader will see a novel method and composition of food and tobacco ingredients used for weight loss and smoking cessation which has the following advantages: it is virtually free of negative side effects, can safely and successfully be combined with other existing and future methods and medications. In addition, it is inexpensive and easy to use.

Although the present invention has been described in connection with the preferred embodiments, the invention should not be limited. Many variations are possible. For example, the ingredients comprising the composition may be slightly varied.

1. A processes for causing weight loss or avoidance of weight gain in mammals, comprising oral administration to said mammals of a composition of “cellulose ether” (methocel) and alcohol delivered in pharmaceutical dosage form such as capsules, tablets, and troche, whereby all of the above ingredients together effectively diminish appetite.

2. The composition of claim 1 wherein alcohol is added to the above ingredient of cellulose ether in the liquid form and then evaporated, resulting in a powder.

3. The composition of matters in claim 1 wherein the content of the dosage further including protein.

4. The composition of matters in claim 3 wherein the content of the dosage further including condiments and sweeteners.

5. The composition of matters in claim 4 wherein the content of the dosage further including caffeine.

6. The composition of the matters in claim 5 wherein the contents of the dosage are delivered in the form of chewing gum.

7. The composition of the matters claim 5 delivered in the form of a small tablet to be placed in the oral cavity, to be absorbed without the process of swallowing.

8. The composition of the matters in claim 5 delivered in the form of the paste to be specifically applied to the designated parts of the tongue.

9. The composition of condiments, tobacco, extracts and alcohol presented in liquid form to be dispensed in the oral cavity in a form of drops, whereby aiding individuals to stop smoking.

10. The composition of matters in claim 8 specifically applied to the designated parts of the tongue and on the part of the cigarette which is placed in the mouth, encouraging patient to smoke and inhale through the thick paste, whereby said patient develops aversion to smoking.
